(19) United States
(12) Patent Application Publication
(10) Pub. No.: US 2022/0241477 A1
(43) Pub. Date: Aug. 4, 2022

(54) PORTABLE CONTINUOUS RENAL REPLACEMENT THERAPY SYSTEM AND METHODS

(71) Applicant: Victor Gura, Beverly Hills, CA (US)

(72) Inventor: Victor Gura, Beverly Hills, CA (US)

(21) Appl. No.: 17/650,006

(22) Filed: Feb. 4, 2022

Related U.S. Application Data
(60) Provisional application No. 63/145,695, filed on Feb. 4, 2021.

(51) Int. Cl. A61M 1/16; A61M 1/26; A61M 1/36; A61M 39/28; B01J 20/22; B01J 20/02; B01J 20/20

(52) U.S. Cl. CPC A61M 1/1631; A61M 1/267; A61M 1/3626; A61M 39/281; B01J 20/223; B01J 20/0211; B01J 2220/66; A61M 2205/8206; A61M 2205/15; A61M 2205/18; A61M 2205/3303; A61M 2209/06; B01J 20/20

(57) ABSTRACT

Various embodiments disclosed relate to a portable system for continuous renal replacement therapy. The present disclosure includes a system including a dialyzer, a blood circuit, a dialysate circuit, a cannister, a pump, and a housing. The housing can encase the system, including the dialyzer, circuits, cannister and pump. The system can be transformed between an active transport mode and a stationary mode. In the active transport mode, the components can be within the housing, allowing for patient mobility while attached to the system.

### PRIORITY APPLICATION

This application claims priority to U.S. Provisional Patent Application 63/145,695, filed on Feb. 4, 2021, the disclosure of which [is/are] incorporated by reference herein in [its/their] entirety.

### BACKGROUND

Continuous renal replacement therapy (CRRT) can be used to provide renal support to patients experiencing acute kidney injuries, and patient who are hemodynamically unstable. Acute kidney injuries can be common complications in critically ill patients, or occur in injured patients in austere environments, such as a battlefield, combat scenario, or other transitory environments.

### SUMMARY OF THE DISCLOSURE

Additional aspects and advantages of the present disclosure will become readily apparent to those skilled in this art from the following detailed description, wherein only some examples of the present disclosure are shown and described, simply by way of illustration of the several modes or best mode contemplated for carrying out the present disclosure. As will be realized, the present disclosure is capable of other and different examples, and its several details are capable of modifications in various obvious respects, all without departing from the disclosure. Accordingly, the drawings and description are to be regarded as illustrative in nature, and not as restrictive.

Disclosed herein is a system and methods for continuous renal replacement therapy (CRRT). The portable CRRT (PCRRT) system is transportable for use in any environment, such as including austere environments, and can run on portable power with a lesser volume of water compared to conventional therapies. This battery operated; sorbent-based machine can replace renal function in critically ill patients with acute renal failure.

The device can circulate the patient's blood through the lumen of hollow fibers of a dialysis filter. The system can use counter-current flow. The system can include a bubble detector, blood clamp, blood leak detector, wetness sensor, and ammonia sensor.

The PCRRT system can operate based on a variety of power sources, such as portable power sources, which can provide power during loss of main external AC power and during active transport. The PCRRT system can have two modes, an active transport mode where it is packed into a carry-ready case, and a stationary mode where it can be set up for use. Additionally, the PCRTT system can use a lower volume of water comparative to current commercially available devices for CRRT.

Discussed herein, the PCRRT system can be used to provide renal replacement therapy (RRT) to acute kidney injury (AKI) patients, such as in austere environments where resources to perform RRT are otherwise lacking or absent.

RRT can replace normal blood filtering functions of the kidneys. RRT can be used, for example, in patients with kidney failure, such as acute kidney injury (AKI) or chronic kidney disease. RRT can include dialysis (hemodialysis peritoneal dialysis), hemofiltration, hemodiafiltration, or kidney replacement. RRT, is a process of purifying the blood of a person whose kidneys are not working normally.

Historically, the rate of severe AKI requiring RRT has risen during conflicts. For example, in the Korean War, 1 in 200 combat injuries was a casualty, and about 25% to about 78% of patients who died of wounds (DOW) may have had AKI treatable by RRT. Advances in RRT technology have potentially reduced mortality in patients on RRT from about 60% to 70% during the Korean war, to about 40% to 50% in the recent Iraq and Afghanistan conflicts. Overall, a readily deployable RRT capability could decrease the DOW rate by about 14% to 40% in austere settings.

Kidneys, when functioning normally, balance the patient pH and electrolyte levels, and remove excess fluid. RRT therapies aim to do the same and supplement or replace these functions, such as in an instance of massive trauma to the kidneys (e.g., an AKI). Thus, devices and methods are desired to provide such kidney functions to critically ill patients, such as patients with unstable blood pressure and electrolyte derangements.

Commonly used dialysis machines treat a patient for three to four hours. These types of machines are difficult to use with patients who cannot tolerate blunt hemodynamic, acidity, and electrolyte balance challenges. For this reason, continuous RRT (CRRT) can be used. CRRT is a slower type of dialysis that puts less stress on the heart compared to other types of RRT. Instead of dialysis over a few hours, CRRT can be done 24 hours a day to slowly and continuously clean out waste products and fluid from the patient. CRRT can include special anticoagulation to keep the dialysis circuit from clotting.

CRRT machines can use three to five times less blood flow than a regular dialysis machine. CRRT machines can function continuously, 24 hours a day akin to biological kidneys, instead of filtering blood for a few hours at a time. The CRRT machines, however, can still require many gallons of water and a large power supply in order to function.

Current forms of CRRT can have a number of disadvantages, such as restricted independence, as people undergoing this procedure cannot travel around due to supply availability and being tethered to a large, stationary device during treatment; high water quality requirements; large water quantity requirements; and the need for a continuous source of electricity, typically provided by a power plug connected to an outlet; requires reliable technology like dialysis and CRRT machines; requirements for a healthcare provider such as nurses or technicians who have more knowledge of the complicated procedure and equipment; and requires ongoing and repetitive time to set up and clean dialysis machines.

Because of the continuous and longer nature of CRRT, use of CRRT in austere environments, such as during conflicts, battlefields, disaster or changing environments, CRRT is typically unavailable unless the patient can be rapidly evacuated from the scene of the conflict. In some cases, in these environments, damage control resuscitation (DCR) or damage control surgery (DCS) could potentially be used. However, future conflicts may have limited evacuation capabilities, and reduced DCR or DCS capacities.

Specifically, CRRT use in austere environments is limited by access to electricity and fresh, clean water, used during CRRT treatment. In some cases, batteries or generators can be used, but may not be adequate. Moreover, large sources of clean water can be difficult to obtain in conflict zones and catastrophe areas. A CRRT machine requires not only fresh, but sterilized water, which can be more difficult to obtain. A reduced required amount of sterile water is desired for an easily deployable portable CRRT machine.

In an example, a portable system for continuous renal replacement therapy is provided. The system can include a dialyzer, a blood circuit, a dialysate circuit, a cannister, a pump, and a housing. The blood circuit can be configured to receive blood from a patient, circulate the blood through the dialyzer, and return cleaned blood to the patient. The dialysate circuit can be configured to circulate dialysate through the dialyzer and remove impurities from the blood. The cannister can include at least one sorbent, the cannister fluidly connectable to the dialysate circuit, wherein the cannister is configured to remove impurities from the dialysate. The pump can be fluidly coupled to the blood circuit and the dialysate circuit, the pump configured to simultaneously drive the blood and the dialysate through the dialyzer countercurrent flow. The housing can encase the system, including the dialyzer, circuits, cannister and pump. The system can be transformed between an active transport mode and a stationary mode. In the active transport mode, the components can be within the housing, allowing for patient mobility while attached to the system.

A method of performing continuous renal replacement therapy while a patient is being transported or in austere environment, the method comprising: continuously removing toxins from blood with a portable system situated on the patient's body, wherein removing toxins comprises using a portable continuous renal replacement therapy system, configured to be transformed between an active transport mode allowing for patient mobility while attached to the system, and a stationary mode for use while the patient is stationary.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1B illustrate schematic diagrams of a PCRRT system in an example.

### DETAILED DESCRIPTION

While some examples of the invention have been shown and described herein, it will be obvious to those skilled in the art that such illustrations are provided by way of example only. Numerous variations, changes, and substitutions will now occur to those skilled in the art without departing from the invention. It should be understood that various alternatives to the examples of the invention described herein may be employed in practicing the invention.

The present disclosure describes, among other things, a portable continuous renal replacement (PCRRT) system and methods. The PCRRT system can include a dialyzer, a blood circuit, a dialysate circuit, a cannister, a pump, and a housing. The housing can encase the system, including the dialyzer, circuits, cannister and pump. The system can be transformed between an active transport mode and a stationary mode. In the active transport mode, the components can be within the housing, allowing for patient mobility while attached to the system.

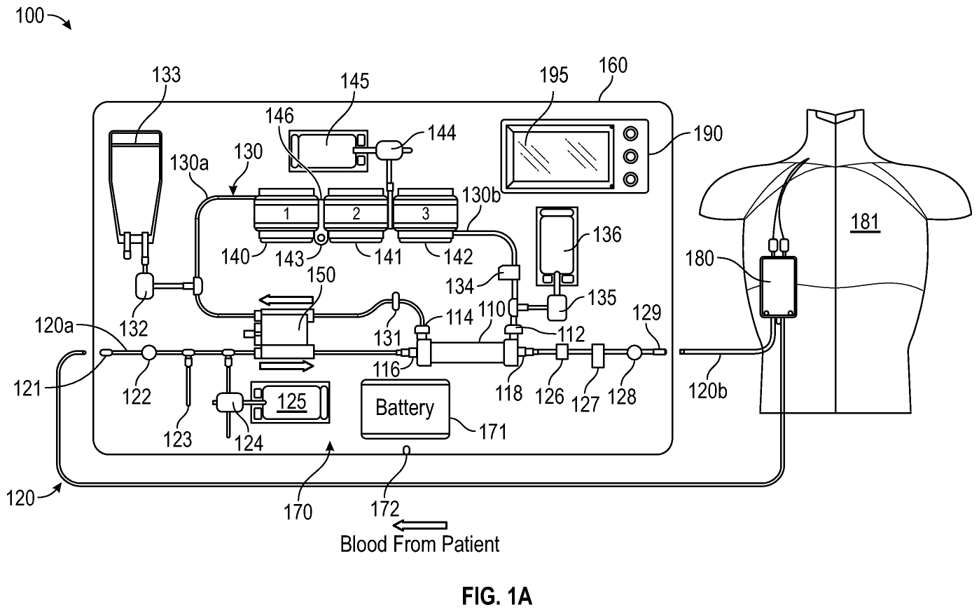

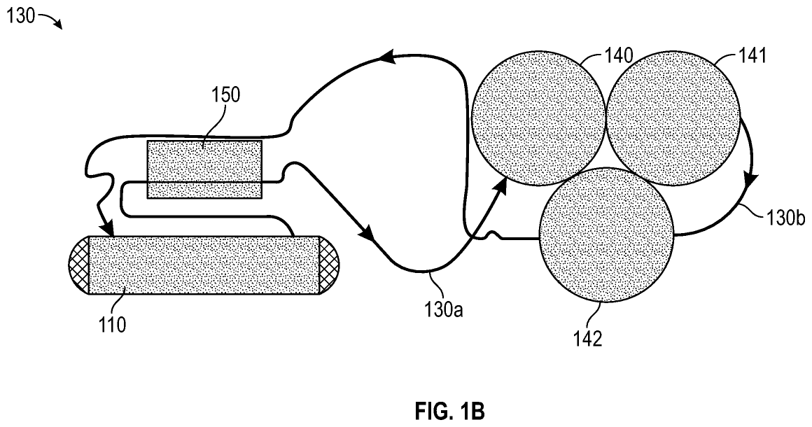

FIGS. 1A-1B illustrate schematic diagrams of a PCRRT system 100 in an example. The system 100 can include a dialyzer 110, a blood circuit 120, a dialysate circuit 130, cannisters 140, 141, 142, a pump 150, a housing 160, a power source 170, a wetness sensor 180, and a control unit 190.

The dialyzer 110 can have a dialysate input 112, a dialysate output 114, a blood inlet 116, and a blood outlet 118. The blood circuit 120 can include an inlet 121, a blood clamp 122, a saline flush 123, auxiliary pump 124, a heparin source 125, a bubble detector 126, a flow sensor 127, a blood clamp 128, and an outlet 129. The dialysate circuit 130 can include a blood detector 131, an auxiliary pump 132 and ultrafiltrate collector 133, a flow sensor 134, and an auxiliary pump 135 with electrolyte source 136. The cannisters 140, 141, 142 can be on the dialysate circuit 130, have one or more air vents 143, and be connected to an auxiliary pump 144 and sodium bicarbonate source 145, in addition to one or more ammonia sensors 146. The pump 150 can be fluidly coupled to both the blood circuit 120 and the dialysate circuit 130. The housing 160 can encircle the other components. The power source can include a battery 171 and an AC power jack 172. The wetness sensor 180 can be coupled to the patient 181, the blood circuit 120, and the dialysate circuit 130.

In system 100, two separate flow channels, one for blood (blood circuit 120) and one for dialysate (dialysate circuit 130), are present. Both blood and dialysate can be propelled through the dialyzer 110 by a double channel pulsating pump 150. The sorbents in the cannisters 140, 141, 142, can continuously cleanse and regenerate dialysate that is circulated through the dialyzer 110. The patient's blood can be propelled through the dialyzer 110, which can be a hollow fiber dialyzer. The dialysate can be pumped through the dialyzer 110 in a flow direction opposite that of the patient's blood. Toxins and fluid in the blood, which are normally removed by the kidneys, can pass through pores of fiber walls in the dialyzer to the dialysate. The toxins and fluid can be eliminated when the dialysate is re-circulated through the sorbent cannisters 140, 141, 142. The opposite phase flow can be done in a pulsatile manner to more efficiently allow exchange of molecules between the dialysate and blood.

The dialyzer 110 can be fluidly coupled to both the blood circuit 120 and the dialysate circuit 130. Dialysate can flow through the dialyzer 110 in a first direction while the blood flows through the dialyzer 110 in a counter current flow. Counter current flow can maximize the gradient between the dialysate circuit 130 and the blood circuit 120, therefore maximizing exchange across the dialyzer 110 membrane. Toxins from the blood flow can diffuse into the dialysate across semi-porous membranes of the dialyzer 110 as the blood and dialysate flow across opposing surfaces of the semi-porous membranes. In an example, blood flow can travel in a clockwise fashion through the blood circuit 120, while the dialysate can flow in a counterclockwise fashion through the dialysate circuit 130.

In system 100, toxins can be removed at a steady rate over a 24-hour period. There are two types of toxins: those bound to protein; and free toxins. Free toxins are generally considered to be more toxic. Examples of toxins that require removal over 24 hours include p-cresyl and indoxyl sulfate. These are part of a group of toxins called protein bound toxins (P-BUTS). The free form, which is the only toxic one, comes out in the urine, keeping its level low in a healthy patient. In dialysis the free fraction comes out on dialysis and the level of the free toxin is also low, however, as soon as the patient is on a dialysis machine, the protein bound toxins re-equilibrate with the free fraction, that comes up again to toxic levels. There are about 25 known P-BUTS. The system 100 can be configured to remove toxins at a steady rate over a 24-hour period.

The blood circuit 120 can be configured to receive blood from a patient, circulate the blood through the dialyzer 110, and return cleaned blood to the patient. The blood circuit 120 can include an inlet 121, a blood clamp 122, a saline flush 123, an auxiliary pump 124, a heparin source 125, a bubble detector 126, a flow sensor 127, a blood clamp 128, and an outlet 129. The blood circuit 120 can include first portion 120a, receiving blood from the patient, and second portion 120b, returning blood to the patient. The first portion 120a can contain un-dialyzed blood, the second portion 120b can contain dialyzed blood. The blood circuit 120 can, for example, be made of tubing or other conduit suitable for flow of blood.

In the first portion 120a of the blood circuit 120, the inlet 121 can be configured for attachment to a patient. Near the inlet, a blood clamp 122 can be configured to allow start and stop of the flow in the blood circuit. A saline flush 123 can additionally be coupled to the blood circuit 120 near the inlet 121 to provide saline.

In some cases, the inlet 121 can be a blood thinner infusion inlet, such as for adding blood thinner to the blood flow to prevent blood clots from forming within the blood circuit 120 of the system 100. In some cases, the blood thinner hookup can be separate from the inlet 121, such as auxiliary pump 124. Such a hookup can be connected to a blood thinner reservoir, such as heparin source 125. Example blood thinners can include heparin, or more specifically, low molecular weight heparins, direct thrombin inhibitors, danaparoid, ancrod, r-hirudin, abciximab, tirofiban and argatroban, among others known to those skilled in the art. Optionally in any example, a blood thinner infusion inlet can be positioned elsewhere on the blood circuit 120, such as after the pump 150. The infusion of one or more blood thinners into the blood circuit 120 can be actuated, for example, by the pump 150.

The first portion 120a of the blood circuit 120 can include flow of blood from the patient that has not yet been treated for toxins. The second portion 120b can include flow of blood back to the patient that has been treated for toxins. In the first portion 120a, the blood circuit 120 can allow for flow of blood from the inlet 121 through the pump 150 to the dialyzer 110 via blood inlet 116, where toxins can move across the fibers in the dialyzer 110 to the dialysate. In the second portion 120b, upon exiting the dialyzer at blood outlet 118, blood can flow towards the outlet 129 towards the patient. The blood flow can run through a number of optional components which may be included in any example, such as the bubble detector 126 and the flow sensor 127.

The bubble detector 126 can be coupled to the blood circuit 120 downstream of the dialyzer 110, such as in second portion 120b. The bubble detector 126 can be configured to detect bubbles in the blood stream, and produce an indication of bubbles if detected. In some cases, the bubble detector 126 can detect specified bubble size in the blood circuit 120. A detection of a bubble can be communicated to the control unit 190 and the user interface 195. The control unit 190 is configurable to pause and/or power off the system 100 upon detection of air bubbles within the blood flow.

The flow sensor 127, can be in line or parallel to the blood circuit 120, such as in second portion 120b of the blood circuit 120. The flow sensor 127 can be configured to measure the rate at which blood is flowing through the system 100. The flow sensor 127 can be a mechanical flow meter, a pressure-based flow meter, a variable area flow meter, an optical flow meter, combinations thereof, or other type of flow sensors.

The flow sensor 127 on the blood circuit 120 can detect the volume of blood moving through the blood circuit over a given time period. This information can be communicated to the control unit 190, which is turn can monitor the flow of blood through the circuit. If the blood flow is outside of a normal range, the control unit 190 can alter the movement of the dialyzer 110 and pump 150 to change the flow of blood and/or dialysate through the system 100. For example, if the blood flow is too slow, it may indicate a clot or blockage, which may need to be addressed. Optionally in any example, a change in flow may trigger an alarm such as an audible, visual, tactile, or other indicia to the user, such as on user interface 195. If the blood flow is too quick, the control unit 190 can slow the mechanism of the pump 150 to modulate the flow of fluid in the system 100 accordingly.

The blood circuit 120 can additionally include a blood clamp 128, configured to activate during a fault state. Activation of the blood clamp 128 can include occlusion of the return portion 120b of the blood circuit 120 to prevent blood from the blood circuit 120 returning to the patient. During a fault state, the blood clamp 128 can trigger to occlude the venous return portion 120b to insulate any related hazard conditions from the patient.

In the blood circuit 120, the blood can be pumped into the dialyzer 110 for removal of toxins. The dialyzer 110 can include dialyzer fibers having lumens, which the blood can be pumped into. Blood clotting can be mitigated by the blood thinner or heparin pumped in at auxiliary pump 124 from the heparin source 125. After circulating through the dialyzer 110, the cleansed blood can be returned to the patient. Blood can flow back to the patient through the outlet 129 of the blood circuit 120.

The dialysate circuit 130 can be configured to circulate dialysate through the dialyzer 110 and remove impurities from the blood. The dialysate circuit 130 can include a blood detector 131, an auxiliary pump 132 and ultrafiltrate collector 133, a flow sensor 134, and an auxiliary pump 135 with electrolyte source 136.

The dialysate circuit can include first portion 130a and second portion 130b. The dialysate circuit 130 can be a sterile dialysate circuit for flow of dialysate therethrough. The dialysate circuit 130 can allow flow of a dialysate through the dialyzer 110 and the pump 150, through the canisters 140, 141, 142, and back to the dialyzer 110. The dialysate circuit 130 can, for example, be made of tubing or other conduit suitable for flow of dialysate.

The first portion 130a of the dialysate circuit 130 can include a blood detection access port connecting the dialysate circuit 130 to the blood detector 131. The blood detection access port can be coupled the blood detector 131, such that presence of blood in the dialysate exiting the dialyzer 110 can be detected. In some cases, breakage in the membranes of the dialyzer 110 can result in blood entering the dialysate flow. The blood detector 131 can be in communication with the control unit 190 such that the control unit 190 will pause and/or power off the system 100 upon detection of blood in the dialysate, or otherwise cause an alarm to be initiated to the user.

Dialysate can be driven by the pump 150 from the dialyzer 110 through dialysate output 114 into the first portion 130a of the dialysate circuit towards the canisters 140, 141, 142. In some cases, the first portion 130a of the dialysate circuit can be connected to the auxiliary pump 132. The dialysate can be driven through the canisters 140, 141, 142, where the sorbents in the canisters treats the dialysate, and then the dialysate flows out to the second portion 130b of the dialysate circuit 130. In the second portion 130b of the dialysate circuit, the dialysate can be driven from the canisters 140, 141, 142 back towards the dialyzer 110, where the dialysate can enter the dialyzer 110 through the dialysate input 112.

The blood detector 131 can be a blood leak detector coupled to the dialysate circuit 130 distal of the dialyzer 110. As such, the blood detector 131 can be configured to monitor and trigger an alarm if blood is detected in the dialysate circuit.

The auxiliary pump 132 and the ultrafiltrate collector 133 can be configured to remove ultrafiltrate from the system and maintain the system volume. The ultrafiltrate from the dialysate can exit the dialysate circuit 130 and can be collected within the ultrafiltrate collector 133 which can be a bag, canister or any other reservoir for collecting the ultrafiltrate. The auxiliary pump 132 can be used to control flow of ultrafiltrate from the dialysate circuit 130 into the ultrafiltrate collector 133. The ultrafiltrate pump 132 can be a micro-pump. Removal of ultrafiltrate can provide removal of water and sodium from the dialysate. For example, the ultrafiltrate removal rate can be maintained at a physiological rate in order to reduce or avoid blunt hemodynamic changes.

Optionally in any example, one or more flow sensors 134 can be alternatively or additionally be on the dialysate circuit for measuring flow of dialysate. The flow sensor 134 can be a mechanical flow meter, a pressure-based flow meter, a variable area flow meter, an optical flow meter, combinations thereof, or other type of flow sensors.

Optionally in any example, the dialysate circuit 130 can include one or more filters, such as to remove particulates from the system.

Optionally in any example, the dialysate circuit 130 can include one or more points at which optional electrolyte is infusible into the dialysate flow. One or more types of optional electrolyte solutions can be added into the dialysate flow to facilitate maintaining electrolyte homeostasis. For example, one or more of optional electrolyte supplement solutions, such as electrolyte supplement solutions comprising sodium bicarbonate, calcium, and/or magnesium, can be infused into the dialysate flow at one or more optional electrolyte infusion points. The electrolyte supplement auxiliary pump can introduce calcium, magnesium and, potentially, potassium into the dialysate after it has passed through the regeneration sorbent cartridges.

Optionally in any example, the second portion 130b of the dialysate circuit can include one or more electrolyte infusion ports proximate auxiliary pump 135 with electrolyte source 136. The electrolyte reservoir can retain an electrolyte solution. Optionally in any example, the electrolyte solution can be used to adjust the pH of the dialysate. The electrolyte solution can be, for example, sodium bicarbonate solution. The electrolyte solution can be infused into the dialysate flow via an electrolyte infusion port. Flow of the electrolyte solution into the dialysate flow can controlled by an electrolyte solution pump. Such an electrolyte solution pump can be configured to pump up to about 5 milliliters per hour (mL/hr), or for example from about 1 mL/hr to about 2 mL/hr, up to about 5 mL/hr.

In the system 100, the dialysate circuit 130 can be configured to run with a smaller volume of dialysate compared to other CRRT systems. For example, the dialysate circuit 130 can be configured to run with about 250 mL to 350 mL of dialysate, or about 300 mL of dialysate. In examples, the system 100 can have a weight of approximately 30 pounds (˜13.6 kilograms) or less.

The cannisters 140, 141, 142 can be on the dialysate circuit 130, have one or more air vents 143, and be connected to an auxiliary pump 144 and sodium bicarbonate source 145. The cannisters 140, 141, 142 can each include at least one sorbent. The cannisters 140, 141, 142 can be fluidly connected the dialysate circuit 130, such as in series. The cannisters 140, 141, 142 can be configured to remove impurities from the dialysate.

The cannisters 140, 141, 142 can include a sorbent that can be, for example, charcoal. Optionally in any example, the cannisters 140, 141, 142 can include a sorbent configured to remove one or more of organic uremic metabolites and heavy metals. Optionally in any example, the sorbent can be configured to remove one or more of creatinine, uric acid and B2 micro globulins, p-cresol, indoleacetic acid and hippurate. In an example, the sorbent can include activated carbon, such as charcoal. The dialysate exiting cannisters 140, 141, 142 can be regenerated dialysate, such that dialysate entering the dialyzer 110 is cleaned dialysate.

The sorbents can be in the form of powders, cartridges, or other suitable variants. In some cases, the sorbents can include multiple layers. The sorbent can include carbon, charcoal, zirconium phosphate, hydrous zirconium oxide, zirconium alloys, organic compounds containing zirconium, inorganic compounds containing zirconium, minerals containing zirconium, urease, or combinations thereof. The various sorbents can, for example, decompose urea into ammonia and carbon dioxide, and adsorb the ammonia while venting carbon dioxide. The various sorbents can, for example remove calcium, magnesium, potassium, and combinations thereof. In some cases, one or more of the sorbents can adsorb phosphorous, creatinine, middle molecules, uremic toxins, and combinations thereof.

The cannisters can, for example, include filters between powder layers. The filters can be made with cellulose materials. This can allow for streamline flow through the sorbents, provide separation between various sorbent material layers, prevent flow channeling between particles of the sorbents, and prevent powder mixing or escaping.

The first cannister 140 can, for example, include immobilized urease and zirconium-phosphate cation exchanger. The second cannister 141 can, for example, include a zirconium-phosphate cation exchanger and hydrous zirconium oxide. The third cannister 142 can include, for example, activated carbon. In some cases, the amounts, types, and orders of sorbents can be changed.

In some cases, the system can include an ammonia sensor 146 configured to monitor ammonia in the system, and configured to trigger an alarm if ammonia is detected above a given threshold. A high level of ammonia in the system can sometimes be an indication that a sorbent, such as a zirconium phosphate, may be saturated and in need of replacement.

Optionally in any example, the dialysate circuit 130 can include one or more bicarbonate sources 145 with an auxiliary pump 144. The auxiliary pump 144 can be initiated to introduce bicarbonate into the dialysate after passing through the cannisters 140 and 141.

The pump 150 can be fluidly coupled to both the blood circuit 120 and the dialysate circuit 130 to move both blood and dialysate through the system. The pump 150 can be configured to simultaneously drive the blood and the dialysate through the dialyzer countercurrent flow.

In some cases, the pump 150 can operate in pulsatile opposite phases. The pump 150 can introduce a pattern that results in more efficient transfer of fluids and molecules across the membrane of the dialysis filter in the dialyzer 110. This can improve clearance of toxins from the blood. The flow pattern can, for example, allow for clearance of uremic toxins, but removal of beta 2 microglobulins and serum phosphorus. In such a pulsatile flow, the pressure and flow in the blood compartment of the filter are at their highest point when, in the dialysate compartment, both flow and pressure are at their lowest points. These opposite high/low points intermittently reverse, creating a “push-pull” traffic across the pores of the dialyzer membrane. This can increase the effectiveness of the convective transfer of molecules and fluid in the dialysis filter, thereby improving the clearance of uremic toxins, despite the miniaturization of the device.

The pump 150 can be a side-to-side pulsatile pump. The side-to-side pulsatile pump 150 can be powered by a battery, including a rechargeable battery, and/or by an electrical wall outlet. For example, the side-to-side pulsatile pump 150 can be powered by a battery to enable transport of the pump 150, thereby facilitating transport of the dialysis system which incorporates the pump 150, such as system 100. An example of such a side-to-side pump is disclosed in U.S. patent application Ser. No. 15/890,718, now issued as U.S. Pat. No. 10,933,183 to Victor Gura; the entire contents of which are incorporated herein by reference.

The side-to-side pulsatile pump 150 can be configured to retain a blood tubing permitting the flow of blood therethrough from the patient, and a dialysate tubing permitting flow therethrough of dialysate, within a pump casing. The pump can include a compression disc configured to provide side-to-side motion to apply a first pressure to the blood ventricle tubing and a second pressure to the dialysate ventricle tubing in alternate fashion. This can allow for alternating pumping of the blood circuit 120 and the dialysate circuit 130. In some cases, the pump can be driven by a motor and gear box. The pump 150 can create a pulsatile flow where the blood pulses are out of phase with the dialysate pulses, such that, for example, the peak of the blood pulse is 90 to 180 degrees out of phase with the peak of the dialysate flow.

One or more side-to-side pulsatile pumps described herein can be configured to provide desired pumping volume for both blood and dialysate, while reducing or eliminating problems associated with known pumps. Optionally in any example, one or more side-to-side pulsatile pumps described herein can provide pumping volumes of greater than about 35 milliliter per minute (mL/min). Optionally in any example, a dialysis system using a side-to-side pulsatile pump can provide a flow rate of dialysate of about 100 mL/min.

The housing 160 can encircle the other components, including the dialyzer, the blood circuit dialysate circuit, the cannister, and the pump. The housing can be a plastic, composite, or metallic material suitable for transporting the system 100. In examples, the housing 160 can comprise a portable container, such as a trunk, backpack, hard case, suitcase and the like, and can include wheels for portability, as well as straps and the like for attaching to a body of a person.

The housing 160 and the system 100 can be configured to be transformed between an active transport mode and a stationary mode. In the active transport mode, the dialyzer, blood circuit, dialysate, and cannister can be encased in the housing. The active transport mode can allow for patient mobility while attached to the system 100. The stationary mode can be for when the patient is stationary.

The power source 170 can be a portable power source, such as a battery or a rechargeable battery, connected to the system 100. In some cases, the power source 170 can additionally include an option to plug into a wall outlet. For example, when the system is in the active transport mode, the portable power source, e.g., battery 171, can be used to provide power to the pump. The portable power source can be portable power source comprises a rechargeable battery. In the stationary mode, the system can plug into a wall outlet comprising an AC power source using AC power jack 172.

The wetness sensor 180 can be coupled to the patient 181, the blood circuit 120, and the dialysate circuit 130. The wetness sensor 180 can be configured to detect fluid leakage between the system and the patient. For example, the wetness sensor 180 can be used to detect fluid leakage between a catheter or cannula used to connect the PCRRT system 100 to a patient. A detected fluid leak can trigger an alarm or fault condition, and pause function of the system 100.

The control unit 190 can be in electrical communication with one or more components of the system 100. For example, the control unit 190 can be in communication with the bubble detector 126 and the blood detector 131 such that an alarm is initiated when air bubbles are detected in the blood flow and/or blood is detected in the dialysate flow. Optionally in any example, the control unit 190 is configured to pause and/or power down the system 100 upon detection of air bubbles in the blood flow and/or blood in the dialysate flow. Optionally in any example, the control unit 190 is configured to control the pump 150 to provide desired flow of blood and/or dialysate through the system 100. The control unit 190 can control one or more optional pumps configured to control flow of electrolyte into the dialysate, blood thinner into the blood flow, and/or ultrafiltrate from the dialysate.

The user interface 195 can allow for the patient to see status updates or monitor functioning of the system 100. The user interface 195 can include, for example, buttons, a screen, lights, or other indicia that can convey whether the system is functioning properly.

A method of performing continuous renal replacement therapy can be done while a patient is being transported or in austere environment. The method can include transporting a patient while the patient is connected to a portable continuous renal replacement therapy system in an active transport mode and continuously removing toxins from blood with the portable continuous renal replacement therapy system while transporting the patient.

The PCRRT can be used to treat patients that suffer from AKI due to injury or illness, in austere environments lacking accessible connections to electrical wall outlets and lesser volumes of sterile fluid. For example, the PCRRT system can be used in battlefield situations where casualties cannot be urgently evacuated, in public health emergencies, or for providing uninterrupted CRRT during active transport of patients on extracorporeal life support in which interruption of CRRT would endanger the patient.

The PCRRT can be a transportable device for use in both a stationary mode and an active transport mode. The PCCRT can be used in the stationary mode, such as at a patient's bedside or in their room. The PCCRT can use a reduced amount of dialysate, such as up to about 300 mL per day, compared to 30-40 liters a day. In the active transport mode, the PCCRT can, for example, be battery operated, conferring mobility to the patient without the need to interrupt treatment. The PCRRT can be used, for example, while actively transporting a patient from one place to another, by gurney, in a vehicle, or during flight.

### Various Notes & Examples

Example 1 can include a portable system for continuous renal replacement therapy, the system comprising: a dialyzer; a blood circuit configured to receive blood from a patient, circulate the blood through the dialyzer, and return cleaned blood to the patient; a dialysate circuit configured to circulate dialysate through the dialyzer and remove impurities from the blood; a cannister comprising at least one sorbent, the cannister fluidly connectable to the dialysate circuit, wherein the cannister is configured to remove impurities from the dialysate; a pump fluidly coupled to the blood circuit and the dialysate circuit, the pump configured to simultaneously drive the blood and the dialysate through the dialyzer countercurrent flow; and housing encasing the system, wherein the dialyzer, the blood circuit dialysate circuit, the cannister, and the pump are integrally disposed in the housing; wherein the system is configured to be transformed between: an active transport mode wherein the dialyzer, blood circuit, dialysate, and cannister are encased in the housing, the active transport mode allowing for patient mobility while attached to the system, and a stationary mode for use while the patient is stationary.

Example 2 can include Example 1, wherein in the active transport mode, the system further comprises a portable power source configured to provide power to the pump.

Example 3 can include any of Examples 1-2, wherein the portable power source comprises a rechargeable battery.

Example 4 can include any of Examples 1-3, wherein in the stationary mode, the system further comprises an AC power source.

Example 5 can include any of Examples 1-4, wherein the dialysate circuit is configured to run with about 250 mL to 350 mL of dialysate.

Example 6 can include any of Examples 1-5, wherein the portable system has a weight of approximately 30 pounds (˜13.6 kilograms) or less.

Example 7 can include any of Examples 1-6, further comprising a bubble detector fluidly coupled to the blood circuit, the bubble detector configured to allow detection of bubbles in the blood circuit and produce an indication of bubbles when detected.

Example 8 can include any of Examples 1-7, further comprising a blood clamp configured to activate during a fault state, wherein activation of the blood clamp comprises occlusion of a return portion of the blood circuit to prevent blood from the blood circuit returning to the patient.

Example 9 can include any of Examples 1-8, further comprising a blood leak detector coupled to the dialysate circuit distal of the dialyzer, wherein the blood leak detector is configured to monitor and trigger an alarm if blood is detected in the dialysate circuit.

Example 10 can include any of Examples 1-9, further comprising a wetness sensor configured to detect fluid leakage between the system and the patient.

Example 11 can include any of Examples 1-10, further comprising an ammonia sensor configured to monitor ammonia in the system, and configured to trigger an alarm if ammonia is detected above a given threshold.

Example 12 can include any of Examples 1-11, wherein the at least one sorbent comprises carbon, charcoal, zirconium phosphate, hydrous zirconium oxide, zirconium alloys, organic compounds containing zirconium, inorganic compounds containing zirconium, minerals containing zirconium, urease, or combinations thereof.

Example 13 can include any of Examples 1-12, further comprising a filter configured to remove particulates from the system, the filter fluidly coupled to the pump and the dialysate circuit.

Example 14 can include any of Examples 1-13, wherein the pump comprises a side-by-side pulsatile pump.

Example 15 can include any of Examples 1-14, wherein the housing comprises a portable case selected from the group comprising a trunk, backpack, hard case and suitcase.

Example 16 can include a method of performing continuous renal replacement therapy while a patient is being transported or in austere environment, the method comprising: transporting a patient while the patient is connected to a portable continuous renal replacement therapy system in an active transport mode; and continuously removing toxins from blood with the portable continuous renal replacement therapy system while transporting the patient.

Example 17 can include Example 16, wherein further comprising converting the portable continuous renal replacement therapy system to a stationary mode, and continuing to remove toxins from blood while in the stationary mode.

Example 18 can include any of Examples 16-17, further comprising powering the portable continuous renal replacement therapy system with a portable energy source.

Example 19 can include any of Examples 15-18, wherein using the portable continuous renal replacement therapy system comprises using about 250 mL to 350 mL of dialysate.

Example 20 can include any of Examples 15-19, wherein using the portable continuous renal replacement therapy system comprises using about 250 mL to 300 mL of dialysate.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A portable system for continuous renal replacement therapy, the portable system comprising:
a dialyzer;
a blood circuit configured to receive blood from a patient, circulate the blood through the dialyzer, and return cleaned blood to the patient;
a dialysate circuit configured to circulate dialysate through the dialyzer and remove impurities from the blood;
a cannister comprising at least one sorbent, the cannister fluidly connectable to the dialysate circuit, wherein the cannister is configured to remove impurities from the dialysate;
a pump fluidly coupled to the blood circuit and the dialysate circuit, the pump configured to simultaneously drive the blood and the dialysate; and
a housing encasing the system, wherein the dialyzer, the blood circuit dialysate circuit, the cannister, and the pump are integrally disposed in the housing;
wherein the system is configured to be transformed between:
an active transport mode wherein the dialyzer, blood circuit, dialysate, and cannister are encased in the housing, the active transport mode allowing for patient mobility while attached to the system, and
a stationary mode for use while the patient is stationary.

2. The portable system of claim 1, wherein in the active transport mode, the system further comprises a portable power source configured to provide power to the pump.

3. The portable system of claim 2, wherein the portable power source comprises a rechargeable battery.

4. The portable system of claim 1, wherein in the stationary mode, the system further comprises an AC power source.

5. The portable system of claim 1, wherein the dialysate circuit is configured to run with about 250 mL to 350 mL of dialysate.

6. The portable system of claim 1, wherein the portable system has a weight of approximately 30 pounds (˜13.6 kilograms) or less.

7. The portable system of claim 1, further comprising a bubble detector fluidly coupled to the blood circuit, the bubble detector configured to allow detection of bubbles in the blood circuit and produce an indication of bubbles when detected.

8. The portable system of claim 1, further comprising a blood clamp configured to activate during a fault state, wherein activation of the blood clamp comprises occlusion of a return portion of the blood circuit to prevent blood from the blood circuit returning to the patient.

9. The portable system of claim 1, further comprising a blood leak detector coupled to the dialysate circuit distal of the dialyzer, wherein the blood leak detector is configured to monitor and trigger an alarm if blood is detected in the dialysate circuit.

10. The portable system of claim 1, further comprising a wetness sensor configured to detect fluid leakage between the system and the patient.

11. The portable system of claim 1, further comprising an ammonia sensor configured to monitor ammonia in the system, and configured to trigger an alarm if ammonia is detected above a given threshold.

12. The portable system of claim 1, wherein the at least one sorbent comprises carbon, charcoal, zirconium phosphate, hydrous zirconium oxide, zirconium alloys, organic compounds containing zirconium, inorganic compounds containing zirconium, minerals containing zirconium, urease, or combinations thereof.

13. The portable system of claim 1, further comprising a filter configured to remove particulates from the system, the filter fluidly coupled to the pump and the dialysate circuit.

14. The portable system of claim 1, wherein the pump comprises a side-by-side pulsatile pump.

15. The portable system of claim 1, wherein the housing comprises a portable case selected from the group comprising a trunk, backpack, hard case and suitcase.

16. A method of performing continuous renal replacement therapy while a patient is being transported or in austere environment, the method comprising:
transporting a patient while the patient is connected to a portable continuous renal replacement therapy system in an active transport mode; and
continuously removing toxins from blood with the portable continuous renal replacement therapy system while transporting the patient.

17. The method of claim 16, wherein further comprising converting the portable continuous renal replacement therapy system to a stationary mode, and continuing to remove toxins from blood while in the stationary mode.

18. The method of claim 16, further comprising powering the portable continuous renal replacement therapy system with a portable energy source.

19. The method of claim 16, wherein using the portable continuous renal replacement therapy system comprises using about 250 mL to 350 mL of dialysate.

20. The method of claim 16, wherein using the portable continuous renal replacement therapy system comprises using about 250 mL to 300 mL of dialysate.
